(19) United States
(12) Plant Patent Application Publication
(10) Pub. No.: US 2018/0263157 P1
(43) Pub. Date: Sep. 13, 2018

(54) Apple tree named 'UEB 42723'

(50) ;

(71) Applicant: Institute of Experimental Botany AS CR, v.v.i. (UEB), Praha (CZ)

(72) Inventors: Radek Cerny, Ricany u Prahy (CZ); Jan Zima, Turnov (CZ); Otto Louda, Pencin u Liberce (CZ); Jaroslav Tupy, Praha (CZ)

(21) Appl. No.: 15/932,513

(22) Filed: Mar. 8, 2018

Related U.S. Application Data
(60) Provisional application No. 62/600,978, filed on Mar. 8, 2017.

(51) Int. Cl. A01H 6/74

(52) U.S. Cl. PLT/175

(57) ABSTRACT

A new and distinct Malus domestica (Borkh.) ornamental variety that exhibits a columnar tree growth type, compact growth habit, V_{ƒ}-resistance against scab, attractive red-purple flowers, greyed-purple young leaves, green mature leaves, and small, red colored, obloid shaped fruits that persist on the tree after the fall of leaves into Winter. The new variety is suitable for home apple growing as solitary trees and can be useful for pollinating purposes.

### CROSS REFERENCE TO RELATED APPLICATION

This application claims the benefit of U.S. Provisional Patent Application Serial No. 62/600,978, filed on Mar. 8, 2017, entitled “Varieties of Apple Trees”’, the entire disclosure of which is incorporated herein by reference.

Botanical classification: Malus domestica (Borkh.)

Varietal denomination: ‘UEB 42723’.

### BACKGROUND OF THE INVENTION

The present invention comprises a new and distinct cultivar of apple tree botanically classified as Malus domestica (Borkh.) and known by the varietal name ‘UEB 42723’.

The new variety is the result of a cross in a planned breeding program between ‘Evereste’ (female parent, unpatented) and ‘Maypole’ (male parent, U.S. Plant Pat. No. 6,184). The cross resulting in ‘UEB 42723’ occurred in the Spring of 2003 at 310 meters above sea level with a mean annual temperature of 7.7° C. and a mean annual precipitation of 680 mm. The purpose of the breeding program was to develop an ornamental apple variety having a unique appearance with a columnar tree growth type that exhibits V_{ƒ}-resistance against scab. The new variety was discovered in 2009 with the first flowering and fruiting of the original seedling in the Czech Republic. Subsequently, the new variety was asexually reproduced in the Czech Republic by budding/grafting on apple rootstocks in the Spring of 2014.

The new variety is similar to its female parent, ‘Evereste’, in exhibiting small fruits with no russeting present and V_{ƒ}-resistance against scab. However, ‘UEB 42723’ exhibits a columnar tree growth type, red-purple flower petals, and red fruit skin color, while ‘Evereste’ exhibits a ramified tree growth type, white flower petals, and yellow fruit skin color with an orange to red blush present. The new variety is similar to its male parent, ‘Maypole’, in exhibiting a columnar tree growth type, red-purple flower petals, and long fruit stalk. However, ‘UEB 42723’ exhibits longer persisting fruits than ‘Maypole’ that are ovoid in shape and leaves that are ovate in shape with a serrate Type 2 to biserrate margin, while the fruits of ‘Maypole’ are round-conical in shape and its leaves are elongated in shape with a crenate margin.

Further, when compared to apple tree named ‘UEB 42721’ (U.S. Plant Pat. application concurrently applied-for herewith), both varieties exhibit a columnar tree growth type and V_{ƒ}-resistance against scab. However, ‘UEB 42723’ exhibits a compact growth habit and an ovoid fruit shape, while ‘UEB 42721’ exhibits a more erect growth habit and a broadly globose fruit shape.

The following characteristics also distinguish the new variety from other varieties known to the breeders:
Late ripening, ornamental, diploid variety;
Columnar tree growth type;
Compact growth habit with short internodes;
Weak to medium tree vigor;
Red-purple colored flowers;
Early to medium time to beginning of flowering;
Greyed-purple color of young leaves and green tinted color of mature leaves;
Fruit is medium in size and ovoid in shape with slight ribbing;
Long time of fruit persistence; and
Resistance against scab on V_{ƒ}-gene basis.

The new variety has been trial and field tested and has been found to retain its distinctive characteristics and remain true to type through successive asexual propagations.

### DESCRIPTION OF THE DRAWINGS

The accompanying photographic drawings illustrate the new cultivar with the color being as nearly true as possible with color illustrations of this type. It should be noted that colors may vary with growing conditions and time of year:

FIG. 1 illustrates a section of the vertical trunk of the new variety, showing details of its ornamental fruits;

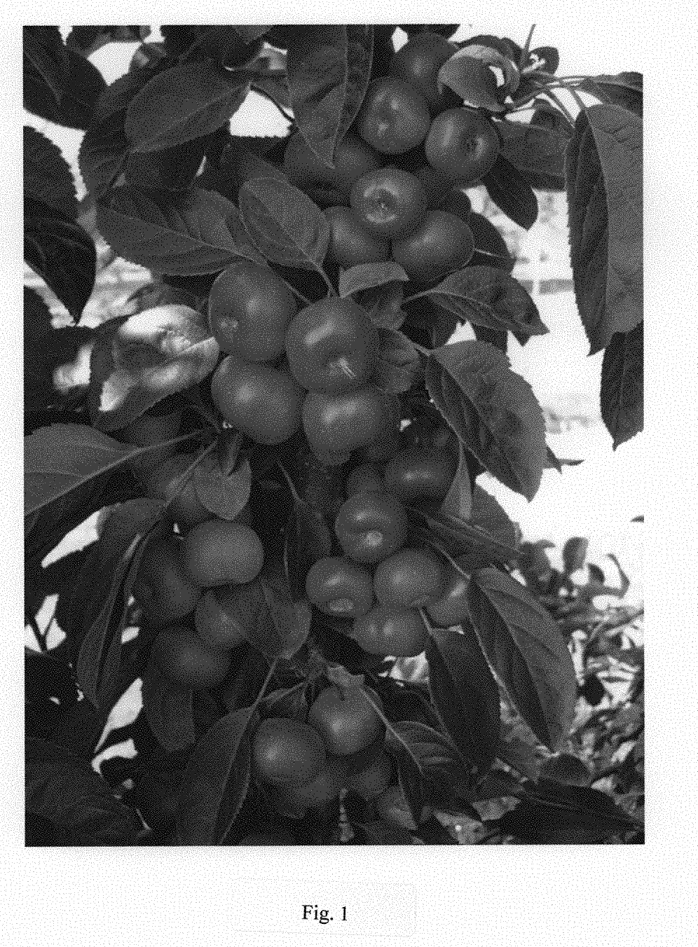

FIG. 2 illustrates details of fruits of the new variety;

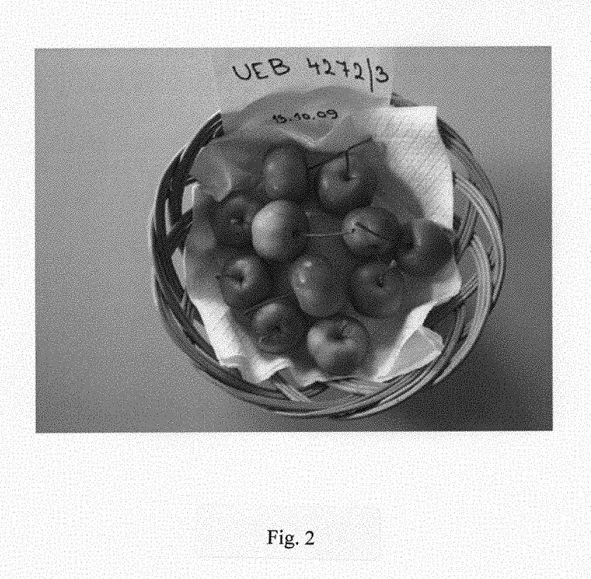

FIG. 3 illustrates a young tree of the new variety grafted on MM 106 rootstock, demonstrating that the fruits are persistent on the tree after its leaves fall into Winter;

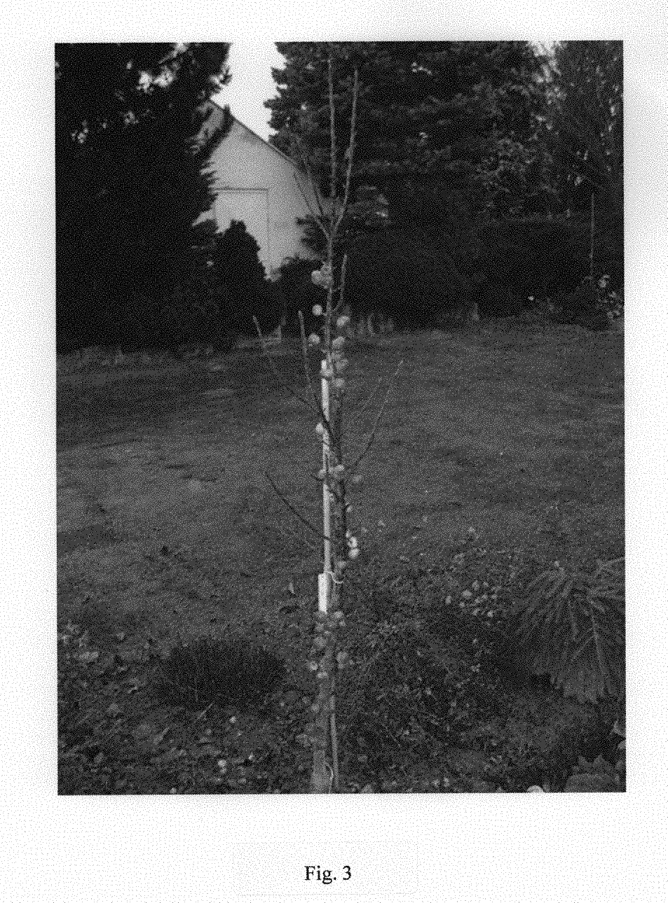

FIG. 4 illustrates a young tree of the new variety grafted on MM 106 rootstock, at first flower opening;

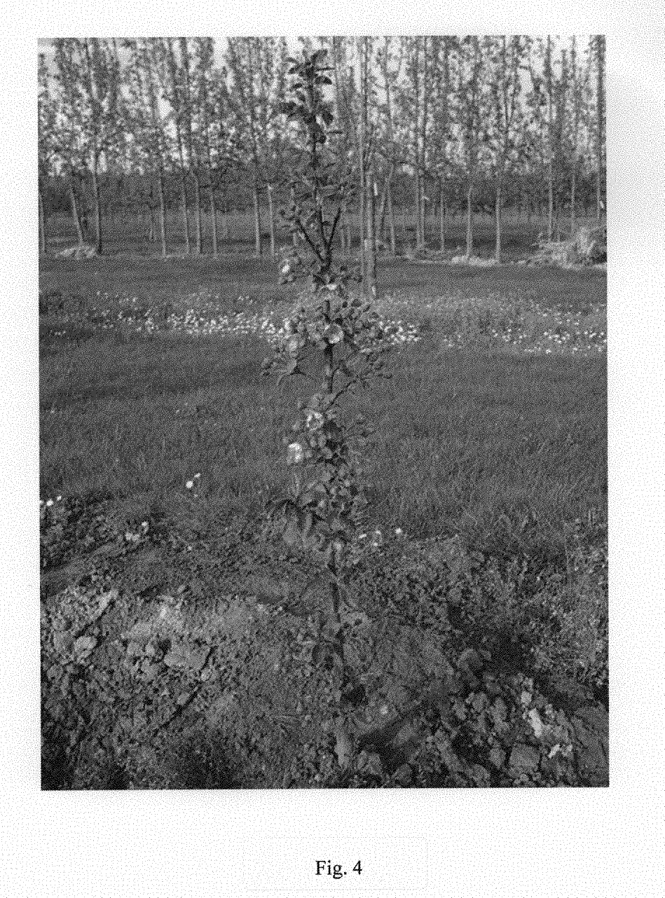

FIG. 5 illustrates a section of the vertical trunk of the new variety, showing predominantly unopened flowers at the beginning of blossoming time;

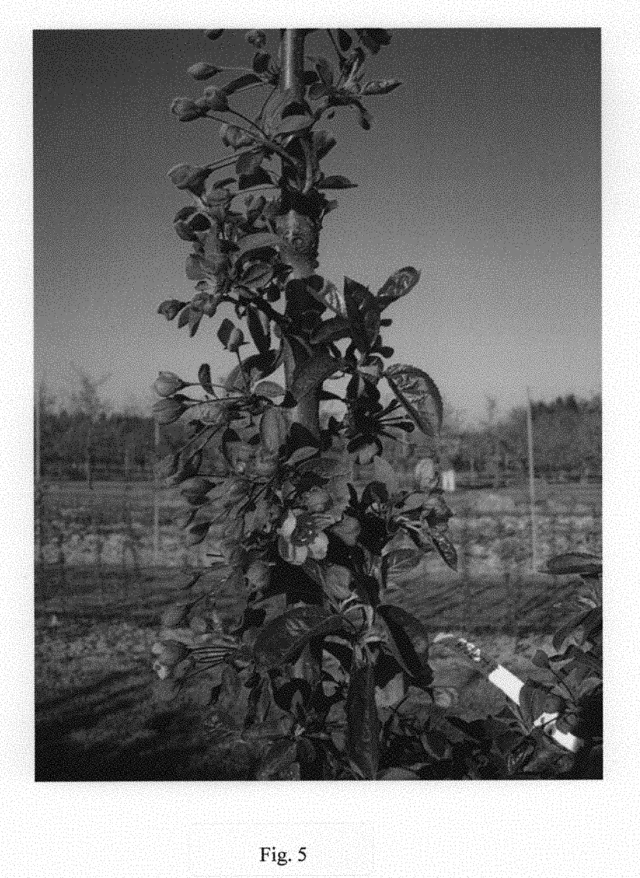

FIG. 6 illustrates a young tree of the new variety grafted on MM 106 rootstock, with fully opened flowers;

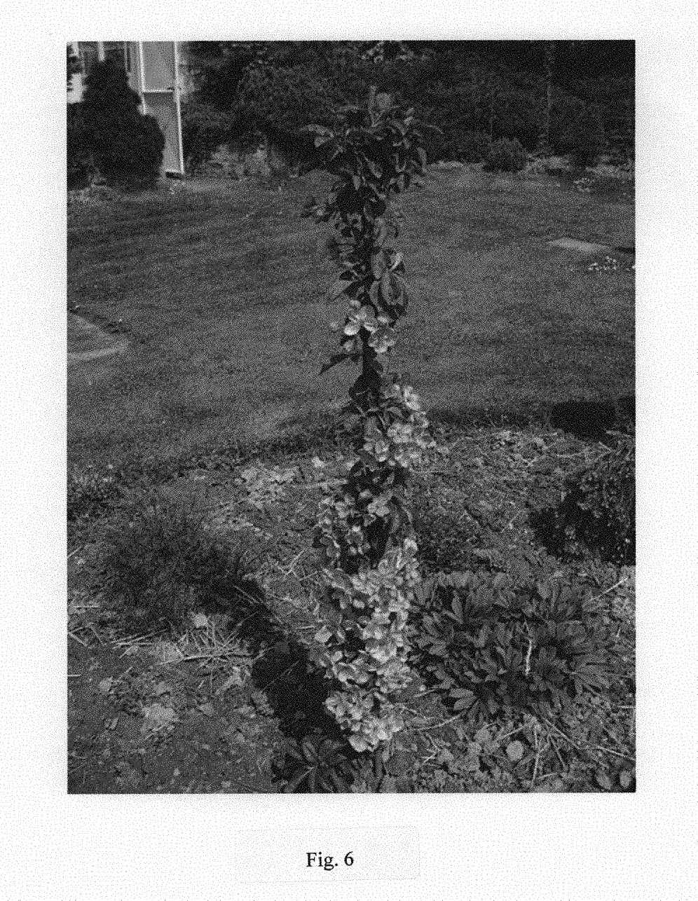

FIG. 7 illustrates details of fully opened flowers of the new variety;

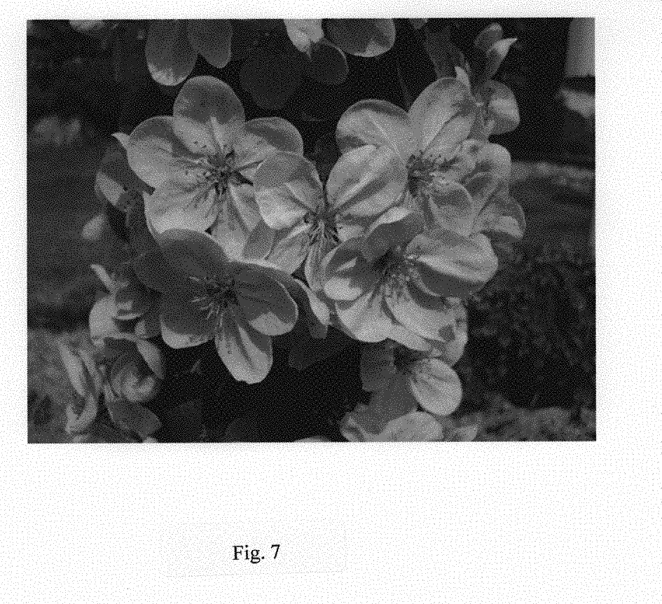

FIG. 8 illustrates a young tree of the new variety grafted on MM 106 rootstock, at the end of blossoming time;

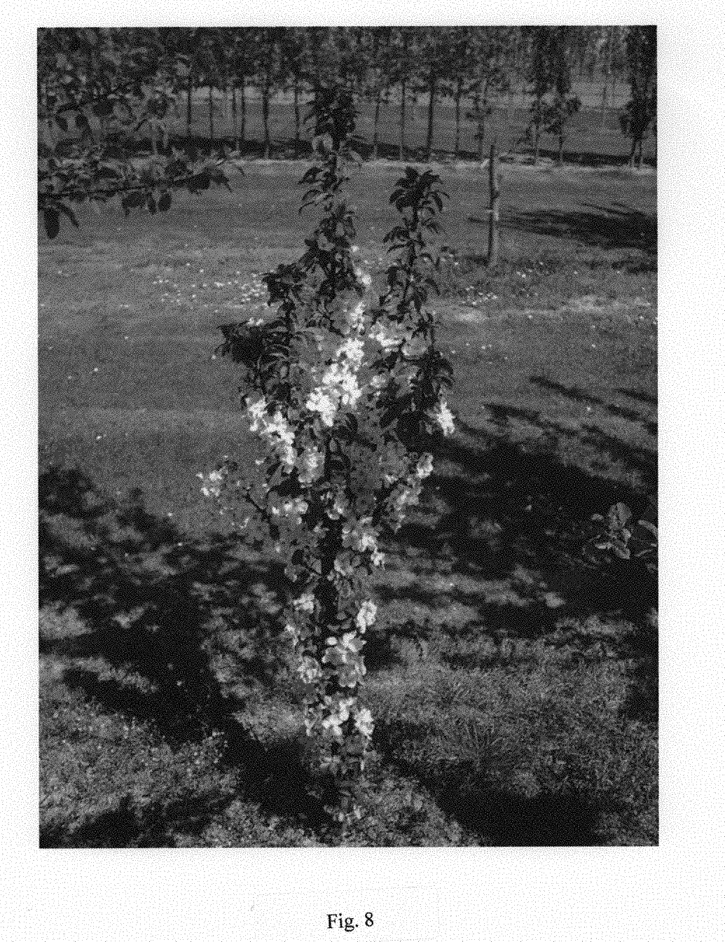

FIG. 9 illustrates a growing shoot of the new variety; and

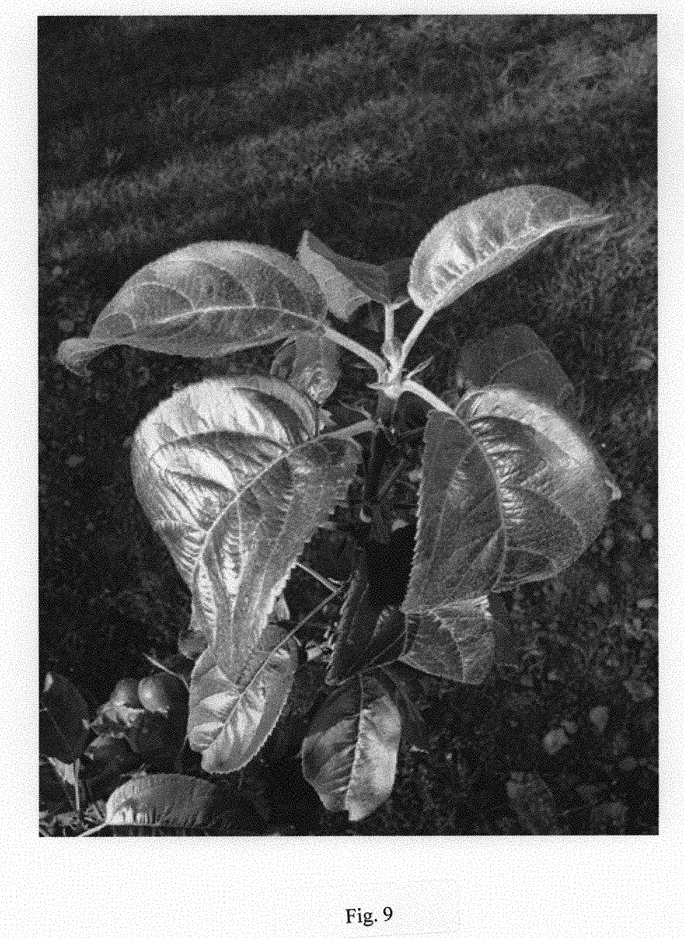

FIG. 10 illustrates a close-up view of a mature leaf and a young leaf of the new variety.

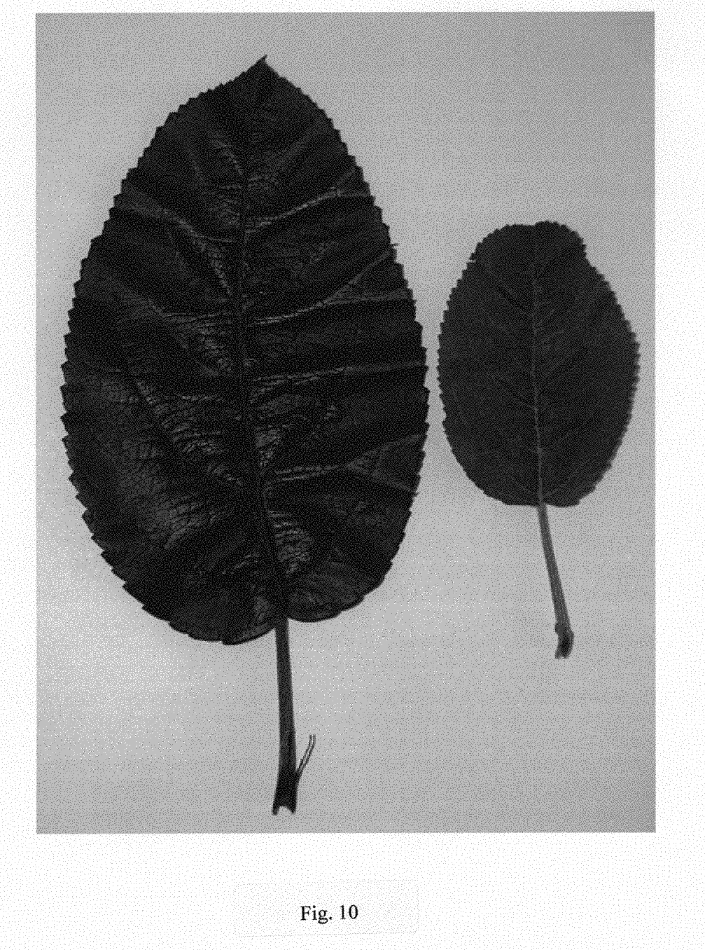

### DESCRIPTION OF THE PLANT

The following detailed description sets forth characteristics of the new cultivar. The new variety was grown under natural field conditions in The Czech Republic. The following fertilizer combination was used (Kg/ha/year): 55 parts nitrogen, 25 parts phosphorous, 60 parts potassium, 55 parts calcium, and 5 parts magnesium. Color references are primarily to The R.H.S. Colour Chart of The Royal Horticultural Society of London and were identified under natural light.
Tree:
   Age.—Approximately 3 years.
   Size.—Crown height of 1.6 m, width of 0.25 m.
   Vigor.—Medium.
   Density.—High.
   Form.—Erect and compact.
   Production.—Very precocious.
   Growth type.—Columnar.
   Bearing.—Annual; predominantly on spurs.
   Trunk.—Size: Approximately 4.0 cm in diameter at 30.0 cm from the soil line. Surface texture: Smooth. Bark color: Greyed-Orange Group RHS 166A. Lenticels (50.0 cm above ground): Length: 1.0 mm to 2.0 mm. Width: 0.5 mm to 1.0 mm. Color: Greyed-Orange Group RHS 164C. Density: Variable, with 5 to 7 lenticels on average per 1 cm².
   Branches.—Not applicable — absent. The variety lacks a conventional branching system along the tree trunk.
   Leaves.—Length: About 60.0 mm to about 86.0 mm, averaging 69.0 mm. Width: About 38.0 mm to about 51.0 mm, averaging about 45.0 mm. Form: Ovate. Texture: Smooth. Thickness: Thin. Base: Rounded and asymmetric. Apex: Acuminate. Margin: Serrate Type 2 to biserrate. Pubescence: Upper surface: None present. Lower surface: Very fine. Color: Young leaves: Upper surface: Greyed-Purple Group RHS 183A. Lower surface: Greyed-Purple Group RHS 183D. Mature leaves: Upper surface: Green Group RHS 139A, combined with some Greyed-Purple Group RHS 187A. Lower surface: Green Group RHS 137C, combined with some Greyed-Purple Group RHS 185C. Petiole: Shape: Straight, with thickening and flattening at the base. Length: About 27.0 mm to 38.0 mm, averaging about 32.0 mm. Diameter: About 1.0 mm in the middle. Color: Red-Purple Group RHS 59A to 59B. Veins: Venation type: Palmate, medium dense. Color: Upper surface: Greyed-Purple Group RHS 186A. Lower surface: Greyed-Purple Group RHS 187A to 187B.
   Flower buds.—Pedicel: Length: Typically in the range of 26.0 mm to 38.0 mm, with an average of 32.0 mm. Diameter: 1.1 mm on average. Color: Predominantly Red-Purple Group RHS 59A. Bud: Length: 14.0 mm on average. Width: 10.0 mm on average. Color: Predominantly Red-Purple Group RHS 59B to 59C.
   Flowers.—Bloom timing: Early to medium. Blooming period: Medium. Pollination requirements: Self-sterile, needs pollinators. Number of flowers per cluster: 5 to 7. Fragrance: Faint. Petals: Number: 5. Length: From 22.0 mm to 28.0 mm, with an average of 25.0 mm. Width: From 20.0 mm to 24.0 mm, with an average of 21.0 mm. Shape: Round to slightly oval. Apex: Obtuse. Base: Mucronate. Aspect: Positioned overlapping. Margin: Entire. Texture and appearance: Soft and smooth. Color: When opening: Upper surface: Red-Purple Group RHS 63B. Lower surface: Red-Purple Group RHS 63B to 63C. Fully opened: Upper surface: Red-Purple Group RHS 62B. Lower surface: Red-Purple Group RHS 62C. Sepals: Shape: Long-conical; pointed. Margin: Entire. Texture: Finely pubescent (colorless). Length: Average of 7.0 mm from the union. Width: 3.0 mm in the middle. Color: Upper surface: Yellow-Green Group RHS 144B, with a Red-Purple Group RHS 59A apex. Lower surface: Red-Purple Group RHS 59B, with a with a Red-Purple Group RHS 59A apex. Stamens: Number (per flower): Average of 19. Filament length: 6.0 mm to 9.0 mm. Anthers: Shape: Oval. Length: 2.0 mm. Color: Yellow Group RHS 10C. Pollen: Color: Yellow Group RHS 10B. Amount (generally): Medium to high. Pistils: Length: 12.0 mm on average. Style: Length: 9.0 mm on average. Color: Red Group RHS 51C. Stigma: Shape: Rounded. Color: Red Group RHS 38A.
   Fruit.—Data currently unavailable.
   Use.—As an ornamental variety having a columnar tree growth type.
   Shipping quality.—Not applicable — ornamental variety.
   Keeping quality.—Not applicable — ornamental variety.
   Tree winter hardiness.—No frost damage observed at the place of origin, lowest winter temperatures approximately −20° C.
   Bud winter hardiness.—No frost damage observed at the place of origin, lowest winter temperatures approximately −20° C.
   Drought tolerance.—Unknown.
   Disease resistance.—V_{ƒ}-resistance against scab.

I claim:

1. A new and distinct variety of Malus domestica (Borkh.) apple tree plant substantially as is herein described and illustrated.
